SEQUENCE LISTING (8 sequences)

SEQ ID NO: 1 - DNA, 47 bp, Artificial Sequence
  Triplex
ttaaaaggggggtttaccccccttttctttgttttccccccttggcc

SEQ ID NO: 2 - DNA, 20 bp, Artificial Sequence
  Triplex-binding
ttttgggcaaggggggaaat

SEQ ID NO: 3 - DNA, 24 bp, Artificial Sequence
  i-motif binding
tttgggttagggttagggttagcg

SEQ ID NO: 4 - DNA, 21 bp, Artificial Sequence
  i-motif
ccctaaccctaaccctaaccc

SEQ ID NO: 5 - DNA, 47 bp, Artificial Sequence
  control Triplex
ttaaaaaaagaaaagaatttattcttttcttctttgttttcttccgg

SEQ ID NO: 6 - DNA, 20 bp, Artificial Sequence
  control Triplex binding
ttttccggaagaaaacaaag

SEQ ID NO: 7 - DNA, 24 bp, Artificial Sequence
  control i-motif binding
tttgggttagggttagggttagcg

SEQ ID NO: 8 - DNA, 21 bp, Artificial Sequence
  control i-motif
cgaggtaatgcaatttgacta

(19) United States
(12) Patent Application Publication
(10) Pub. No.: US 2020/0179522 A1
(43) Pub. Date: Jun. 11, 2020

(54) NANOSTRUCTURES FOR PENETRATING DEEP INTO CANCER TISSUES

(71) Applicant: Onselex Pharmaceuticals, Inc., Wilmington, DE (US)

(72) Inventors: Won Jong Kim, Gyeongsangbuk-do (KR); Jinhwan Kim, Gyeongsangbuk-do (KR)

(21) Appl. No.: 16/295,567

(22) Filed: Mar. 7, 2019

Related U.S. Application Data
(63) Continuation of application No. PCT/KR2018/015536, filed on Dec. 7, 2018.

(51) Int. Cl. A61K 47/54; A61K 47/69; A61K 9/51; C12N 15/113

(52) U.S. Cl. CPC A61K 47/549; A61K 47/6931; B82Y 5/00; C12N 15/113; A61K 47/555; A61K 9/51

(57) ABSTRACT

The present invention relates to nanostructures for penetrating deep into cancer tissues, and more particularly, to nanostructures capable of selectively delivering nanoparticles and drugs to cancer tissues because it is possible to intelligently control the release of the nanoparticles and the drugs depending on the pH condition of the cancer tissues and also having high nanoparticle and drug delivery efficiency because it is possible for the nanoparticles to penetrate deep into the cancer.

### CROSS-REFERENCE TO RELATED APPLICATION

This application is a Continuation of International Application No. PCT/KR2018/015536 filed Dec. 7, 2018, the content of which are hereby incorporated by reference in its entirety.

### TECHNICAL FIELD

The present invention relates to nanostructures for penetrating deep into cancer tissues, and more particularly, to nanostructures capable of selectively delivering nanoparticles and drugs to cancer tissues because the release of the nanoparticles and the drugs may be intelligently controlled depending on the pH condition of the cancer tissues and also having high nanoparticle and drug delivery efficiency because the nanoparticles may penetrate deep into a cancer.

### BACKGROUND ART

Nanoparticles used in the field of drug delivery systems have different properties such as stability in blood circulation, penetration into tissues, and inflow into cells, and the like, depending on their sizes. In particular, the nanoparticles having a size of 50 nm or more and 300 nm or less selectively penetrate loose blood vessels in cancer tissues and remains in the blood vessels for a long period of time (so-called an enhanced permeation and retention (EPR) effect), but have an advantage in that it is possible to selectively deliver the nanoparticles into the cancer tissues because the nanoparticles do not easily penetrate dense blood vessels in normal tissues. However, the nanoparticles having the corresponding diameter have limitations in that the nanoparticles show relatively low penetration into the interior of the three-dimensional (3D) cancer tissues due to their size.

On the other hand, the nanoparticles having a small size of less than approximately 20 nm are effective for penetrating into the interior of the 3D cancer tissues, but have drawbacks in that, when injected into the bloodstream, the nanoparticles may accumulate even inside the normal tissues due to their small size, thereby causing serious side effects.

That is, the nanoparticles having a size of 50 nm or more and 300 nm or less induce their own selective inflow into cancer tissues, but have limitations in that they show low penetration into the interior of the 3D cancer tissues. Also, the nanoparticles having a small size of less than approximately 20 nm less induce their high-level penetration into the cancer tissues, but have drawback in that they have non-specific accumulation in the normal tissues.

Meanwhile, a nucleic acid sequence is merely a biological substance carrying genetic information, and may be used as a polymeric structure having repeats of randomly repeated base sequences consisting of adenine (A), thymine (T), guanine (G), and cytosine (C). They have high biocompatibility, and also show various physicochemical properties, depending on the alignment of the base sequence. In particular, C in the base sequence generally binds to G via a selective hydrogen bond to form a double-helix structure, but it has a higher biding affinity to protonated C at an acidic pH level and has a characteristic of forming a new bond after breaking the original hydrogen bond in the double-helix structure. It is known that a variety of dynamic structural changes are possible through this.

Cancer tissues and cells have their intrinsic pH gradients. In general, it is known that energy is consumed even in the lactic acid cycle due to the rapid metabolism in cancer cells, and thus lactic acid, which is one of by-products, serves so that the surrounding environment of a cancer tissue has a low pH of approximately 6.5. Also, it is known that the inside of cancer cells has a pH value decreased to approximately pH 5.0 in intracellular vesicles (also referred to as endosomes and lysosomes).

Accordingly, there is a need for novel therapeutic agents capable of intelligently controlling the behavior of drug release into the 3D cancer tissues in response to different pH levels in the surroundings of cancer tissues and the inside of the cancer cells.

### DISCLOSURE

### Technical Problem

To solve the above problems, the present inventors have prepared nanostructures which is pH sensitive and can support nanoparticles capable of supporting a drug through coupled induction between nucleic acids, and found that such nanostructures can effectively control the nanoparticle and drug release behaviors depending on the pH of the surrounding environment of cancer, and thus have selectivity for drug delivery and high penetrability into the interior of 3D tissues. Therefore, the present invention has been completed based on these facts. Also, the present inventors have found that the nanostructures can be used to control an anticancer drug release behavior and a therapeutic effect in 3D environments, and induce the cancer tissue-specific inflow of the nanoparticles, thereby maximizing the delivery efficiency and minimizing the side effects.

Therefore, an object of the present invention is to provide nanostructures capable of penetrating deep into cancer tissues and having nanoparticles supported therein, wherein the nanoparticles and the nanostructures have pH sensitivity, and each of the nanoparticles and nanostructures include DNA.

### Technical Solution

To solve the above problems, according to one aspect of the present invention, there is provided nanostructures having nanoparticles supported therein,

wherein the nanoparticles and the nanostructures have pH sensitivity,

each of the nanoparticles and the nanostructures include DNA.

The nanoparticles may have a diameter of 20 nm or less, and the nanostructures may have a diameter of 50 nm or more.

The DNA may include a base sequence in which a ratio of cytosine (C) is in a range of 40% to 80%.

The nanoparticles and the nanostructures may be complementarily bound by means of the base sequence of the DNA.

The nanostructures may include DNA having a triplex base sequence, and the nanoparticles may include DNA having a sequence complementary to the triplex base sequence.

The triplex base sequence may be a base sequence set forth in the following SEQ ID NO: 1, and the sequence complementary to the triplex base sequence may be a base sequence set forth in the following SEQ ID NO: 2.

The nanostructures may have pH sensitivity at pH 6.0 to 6.5.

The nanoparticles may have pH sensitivity at pH 5.5 or lower.

An i-motif DNA base sequence and a sequence complementary thereto may be conjugated onto surfaces of the nanoparticles.

The i-motif DNA base sequence may be a base sequence set forth in the following SEQ ID NO: 3, and the sequence complementary to the i-motif DNA base sequence may be a base sequence set forth in the following SEQ ID NO: 4.

An anticancer drug may be supported in the i-motif DNA base sequence and the sequence complementary thereto.

The anticancer drug may include one or more selected from the group consisting of doxorubicin (DOX), cisplatin, etoposide, paclitaxel, doxetaxel, fluoropyrimidine, oxaliplatin, campthotecan, belotecan, podophyllotoxin, vinblastine sulfate, cyclophosphamide, actinomycin, vincristine sulfate, methotrexate, bevacizumab, thalidomide, erlotinib, gefitinib, camptothecin, tamoxifen, anasterozole, Gleevec, 5-fluorouracil (5-FU), floxuridine, leuprolide, flutamide, zoledronate, vincristine, gemcitabine, streptozotocin, carboplatin, topotecan, irinotecan, vinorelbine, hydroxyurea, valrubicin, retinoic acid, meclorethamine, chlorambucil, busulfan, doxifluridine, vinblastin, mitomycin, prednisone, testosterone, mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, cortisone, corticosteroid, and a combination thereof.

### Advantageous Effects

The nanostructures according to the present invention can intelligently control a nanoparticle or drug release behavior in response to different pH levels, depending on the control of a base sequence.

Also, the nanostructures according to the present invention can show much higher biocompatibility due to the properties of nucleic acids, compared to synthetic polymers, etc., and thus can be used as a drug delivery system.

In addition, the nanostructures according to the present invention can induce the cancer tissue-specific inflow of the nanoparticles, thereby maximizing the delivery efficiency and minimizing the side effects.

### DESCRIPTION OF DRAWINGS

This application file contains at least one drawing executed in color. Copies of this patent or patent application publication with color drawing(s) will be provided by the Office upon request and payment of the necessary fee.

FIG. 1 shows TEM (FIG. 1A) and SEM (FIG. 1B) images of nanostructures according to one exemplary embodiment of the present invention, and also shows the electron energy loss spectroscopy (EELS) analysis results (FIG. 1C).

FIG. 2 shows the results of evaluating the release of nanoparticles and a drug according to a change in pH of the nanostructures according to one exemplary embodiment of the present invention.

FIG. 3 shows the results of evaluating the penetration of the nanostructures according to one exemplary embodiment of the present invention using a 3D spheroid model.

FIG. 4 shows the results of evaluating the penetration of the nanostructures according to one exemplary embodiment of the present invention in a bio-distribution and cancer tissues using an animal model.

FIG. 5 shows the results of evaluating anti-cancer effects of the nanostructures according to one exemplary embodiment of the present invention.

### BEST MODE

The present invention provides nanostructures capable of penetrating deep into cancer tissues and having nanoparticles supported therein, wherein the nanoparticles and the nanostructures have pH sensitivity, and each of the nanoparticles and the nanostructures include DNA.

The nanoparticles may have a diameter of 20 nm or less, and the nanostructures may have a diameter of 50 nm or more.

Each of the nanoparticles and the nanostructures may have DNA bound thereto, and the DNA includes a base sequence in which a ratio of cytosine (C) is in a range of 40% to 80%.

The nanoparticles and the nanostructures may be complementarily bound by means of the base sequence of the DNA, and may be arranged so that the nanoparticles may be encapsulated or supported in the nanostructures having a higher diameter.

When the nanostructures include DNA having a triplex base sequence, the nanoparticles may include DNA having a sequence complementary to the triplex base sequence.

In this case, the triplex base sequence is preferably a base sequence set forth in the following SEQ ID NO: 1, and the sequence complementary to the triplex base sequence is preferably a base sequence set forth in the following SEQ ID NO: 2.

SEQ ID NO: 1:
5′-NH2-TTA AAA GGG GGG TTT ACC CCC CTT TTC TTT
GTT TTC CCC CCT TGG CC
SEQ ID NO: 2:
5′-SH-TTT TGG GCA AGG GGG GAA AT

Because the DNA includes a base sequence in which the ratio of cytosine is in a range of 40% to 80%, cytosine is present in a state in which the cytosine is bound to guanine (G) at a neutral pH or pH 7.4. However, because binding between protonated C and C is induced at an acidic pH, one DNA strand has a lot of cytosine residues with different shapes and positions in a sequence thereof, thereby causing a structural change according to the pH changes in the surrounding environments. That is, because the DNA includes a base sequence in which the ratio of cytosine is in a range of 40% to 80%, the nanostructures may have pH sensitivity. Accordingly, the nanoparticles and the nanostructures may show pH sensitivity.

In this case, when the ratio of cytosine in the DNA is less than 40%, the pH sensitivity may be lowered. On the other hand, when the ratio of cytosine in the DNA is greater than 80%, it may be difficult to induce structural changes according to the different pH conditions. The ratio of cytosine is preferably in a range of 55 to 65%, more preferably 60%, but the present invention is not limited thereto.

Because the nanostructures have a diameter of 50 nm or more, the nanostructures may selectively penetrate loose blood vessels in the cancer tissues, and may stay in the blood vessels for a long period of time. Also, when the nanostructures have a diameter of less than 50, it is difficult to support the nanoparticles, and it is also difficult to release the nanoparticles even when the nanoparticles are supported, thereby making it difficult to support a high concentration of the nanoparticles. In addition, when the nanostructures have a diameter of less than 20 nm, a large amount of the nanostructures accumulates inside the normal tissues due to their small size, resulting in side effects. On the other hand, when the nanostructures have a diameter of 300 nm or more, the nanostructures may not be easily penetrated into the loose blood vessels in the cancer tissues. The preferred diameter of the nanostructures is in a range of 50 nm to 300 nm, but the present invention is not limited thereto.

The nanostructures may be porous to support the nanoparticles. In this case, it is desirable that the diameter of the pores is higher than the diameter of the nanoparticles.

According to one exemplary embodiment of the present invention, when the nanostructures are porous, the specific surface area, the average pore diameter and the porosity of the nanostructures are preferably adjusted according to the diameters of the nanostructures and the nanoparticles.

When the nanostructures have a very small specific surface area, it is not easy to support a high concentration of the nanoparticles. On the other hand, when the nanostructures have a very large specific surface area, the nanoparticles or the drug may not stay in the bloodstream for a long period of time due to an increase in in vivo degradation rate and may be easily released even when exposed to mild physical impacts.

Also, when the average pore diameter of the nanostructures is too small, preparation of the nanoparticles having a diameter smaller than the pore diameter of the nanostructures is required, resulting in a complicated manufacturing process and degraded reproducibility. On the other hand, when the average pore diameter of the nanostructures is too high, the nanoparticles or the drug may be easily released even when exposed to mild physical impacts.

Meanwhile, when the porosity of the nanostructures is too small, it is not easy to support the nanoparticles. On the other hand, when the porosity of the nanostructures is too high, the retention time in the bloodstream may be shortened due to an increase in in vivo degradation rate.

The nanostructures have pH sensitivity at pH 6.0 to 6.5. When the pH of the nanostructures is out of this corresponding pH range, the nanostructures are bound to the nanoparticles. When the pH of the nanostructures is in this corresponding pH range, the nanostructures are detached from the nanoparticles so that the nanoparticles are released. As one example, the DNA conjugated to the nanostructures may be converted into duplexes while breaking a bond to the DNA conjugated to surfaces of the nanoparticles at pH 6.5.

As described above, the pH sensitivity of the nanostructures may be adjusted according to the base sequence of the DNA. As one example, the pH conditions in which the bond of the DNA to the nanoparticles is broken may vary by adjusting the ratio of cytosine in the DNA bound to the nanostructures.

When the nanoparticles have a diameter of 20 nm or less, the nanoparticles may easily penetrate into the 3D cancer tissues, and may be supported in the nanostructures having a diameter of 50 nm or more. Therefore, even when the nanoparticles are injected into the bloodstream, the nanoparticles do not accumulate inside the normal tissues, thereby minimizing the side effects.

Also, the nanoparticles have pH sensitivity because the DNA including a base sequence in which the ratio of cytosine is in a range of 40% to 80% is bound to the nanoparticles. Preferably, the nanoparticles may have pH sensitivity at pH 5.5 or lower. In this case, the nanoparticles show sensitivity at pH levels different from the nanostructures.

Because the nanoparticles and the nanostructures show sensitivity at different pH levels, the nanoparticles and the nanostructures are applicable even when there is a minute difference in pH between the surrounding and internal environments of the cancer tissues. As one example, when the pH of the surrounding environment of the cancer tissues is 6.5, the nanostructures respond to release the nanoparticles or the drug. The nanoparticles respond to release the drug in the inside of the cancer cells at pH 5, the value of which is lower than pH 6.5.

Because the nanoparticles and the nanostructures have slightly different pH sensitivity, the nanostructures may effectively control the nanoparticle and drug release behaviors and enhance selectivity in the drug delivery.

The nanoparticles preferably have pH sensitivity at pH 5.5 or lower, but the present invention is not limited thereto.

An i-motif DNA base sequence and a sequence complementary thereto may be conjugated to surfaces of the nanoparticles. In this case, the i-motif DNA base sequence has a property to form an anti-parallel qudruplex structure at approximately pH 5.0 and break an original bond. Also, a structural change in endolysosomes present in the cancer cells may be induced by conjugating the i-motif DNA base sequence and the sequence complementary thereto to surfaces of the nanoparticles.

According to one exemplary embodiment of the present invention, the i-motif DNA base sequence may be a base sequence set forth in the following SEQ ID NO: 3, and the sequence complementary to the i-motif DNA base sequence may be a base sequence set forth in the following SEQ ID NO: 4.

SEQ ID NO: 3:
5′-SH-TTT GGG TTA GGG TTA GGG TTA GCG
SEQ ID NO: 4:
5′-CCC TAA CCC TAA CCC TAA CCC

Also, an anticancer drug may be supported in the i-motif DNA base sequence and the sequence complementary thereto. More specifically, when the anticancer drug is supported in the i-motif DNA base sequence and the sequence complementary thereto, which are conjugated to the surfaces of the nanoparticles, and injected in vivo, the base sequence may show pH sensitivity in the endolysosome. As a result, a double-helix structure may be ultimately converted into a single-stranded structure while the base sequence is being converted into an anti-parallel quadruplex structure in order to release the supported drug. Therefore, the drug may be selectively released in the cells.

The anticancer drug may include one or more selected from the group consisting of doxorubicin (DOX), cisplatin, etoposide, paclitaxel, doxetaxel, fluoropyrimidine, oxaliplatin, campthotecan, belotecan, podophyllotoxin, vinblastine sulfate, cyclophosphamide, actinomycin, vincristine sulfate, methotrexate, bevacizumab, thalidomide, erlotinib, gefitinib, camptothecin, tamoxifen, anasterozole, Gleevec, 5-fluorouracil (5-FU), floxuridine, leuprolide, flutamide, zoledronate, vincristine, gemcitabine, streptozotocin, carboplatin, topotecan, irinotecan, vinorelbine, hydroxyurea, valrubicin, retinoic acid, meclorethamine, chlorambucil, busulfan, doxifluridine, vinblastin, mitomycin, prednisone, testosterone, mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, cortisone, corticosteroid, and a combination thereof. Doxorubicin is preferred, but the present invention is not limited thereto.

A method of adjusting the sizes and shapes of the nanostructures and the nanoparticles may be realized by selecting or altering factors such as the type of structure-directing agents for nanostructures, components of the nanoparticles, a regulatory DNA base sequence, and other factors such as a reaction time required to prepare the nanostructures, an amount of metal ions in the nanoparticles, a reaction terminator added, etc.

According to another exemplary embodiment of the present invention, a pharmaceutical composition including the nanostructures may be provided.

The pharmaceutical composition refers to a composition that is administered for certain purposes. For this purpose of the present invention, the pharmaceutical composition of the present invention may be used to treat diseases or disorders associated with cancer, and may include proteins involved in this treatment, and a pharmaceutically acceptable carrier, excipient or diluent.

The “pharmaceutically acceptable” carrier or excipient refers to carriers or excipients that have been approved by the regulation division of the government or are listed in the pharmacopoeias approved by the government or generally approved for use in vertebrates, more particularly humans.

The pharmaceutical composition may be in the form of a suspension, a solution or an emulsion contained in an oily or aqueous carrier so that the pharmaceutical composition can be suitable for parenteral administration, and may also be prepared in the form of a solid or a semisolid. In this case, the pharmaceutical composition may include a formulating agent such as a suspending agent, a stabilizing agent, a dissolving agent, and/or a dispersing agent. This form may be sterilized, and may be a liquid. This may be stabilized under the manufacturing and storage conditions, and may be preserved against an action of contamination of microorganisms such as bacteria or fungi. Alternatively, the pharmaceutical composition may be in the form of a sterile powder for the purpose of reconstitution with a proper carrier before use. The pharmaceutical composition may be present in a unit dose form, or present in microneedle patches, ampoules, or other unit-dose or multiple-dose containers. Also, the pharmaceutical composition may be stored in a lyophilized (freeze-dried) state requiring simple addition of a sterile liquid carrier, for example, water for injection right before use. An immediate injectable solution and a suspension may be prepared in the form of sterile powders, granules or tablets.

In some non-limiting embodiments, the pharmaceutical composition of the present invention may be formulated into liquids, or may be included in the form of microspheres in the liquids. In any non-limiting embodiments, the pharmaceutical composition of the present invention includes a pharmaceutically acceptable compound and/or mixture at a concentration of 0.001 to 100,000 U/kg as an active ingredient of the present invention. Also, in any non-limiting embodiments, the excipient suitable for the pharmaceutical composition of the present invention includes a preservative, a suspending agent, a stabilizing agent, a dye, a buffer, an antimicrobial agent, an antifungal agent, and an isotonic agent (for example, sugar or sodium chloride). As used herein, the term “stabilizing agent” refers to a compound that is optionally used in the pharmaceutical composition of the present invention in order to increase the storage life. In non-limiting embodiments, the stabilizing agent may be a sugar, an amino acid, a compound, or a polymer. The pharmaceutical composition may include one or more pharmaceutically acceptable carriers. The carrier may be a solvent or a dispersion medium. Non-limiting examples of the pharmaceutically acceptable carrier include water, saline, ethanol, polyols (e.g., glycerol, propylene glycol and liquid polyethylene glycol), oils, and proper mixtures thereof. Also, a parenteral formulation may be sterilized. Non-limiting examples of sterilization techniques include filtration through bacteria-suppressing filters, terminal sterilization, combination of sterile preparations, irradiation, sterile gas spraying, heating, vacuum drying, and lyophilization.

According to one exemplary embodiment of the present invention, the term “administration” refers to the introduction of the pharmaceutical composition of the present invention to a patient using any proper methods. In this case, the pharmaceutical composition of the present invention may be administered via any general route of administration so long as the pharmaceutical composition can reach a target tissue. The pharmaceutical composition of the present invention may be administered by oral administration, intraperitoneal administration, intravenous administration, intramuscular administration, subcutaneous administration, intradermal administration, intranasal administration, intrapulmonary administration, intrarectal administration, intracavitary administration, intraperitoneal administration, intradural administration, and the like. However, the pharmaceutical composition including the nanostructures of the present invention may be administered as an injectable preparation because the pharmaceutical composition is applicable to parenteral administration.

A method of treating a disease using the pharmaceutical composition of the present invention may include administering a pharmaceutically effective amount of the pharmaceutical composition. In the present invention, the effective amount may be adjusted according to various factors including the type of a disease, the severity of a disease, the types and contents of active ingredients and other components contained in the composition, the type of a formulation, and the age, weight, general health condition, sex, and diet of a patient, an administration time, a route of administration, and a secretion rate of a composition, a treatment period, and drugs used together.

### MODE FOR INVENTION

Hereinafter, preferred embodiments of the present invention are provided to aid in understanding the present invention. However, it should be understood that the detailed description disclosed herein is given by way of illustration of the present invention only, and thus various changes and modification may be made without departing from the sprite and scope of the present invention. Also, it will be apparent that such changes and modifications fall within the appended claims.

### Preparative Example 1: Preparation of Nanostructures

### Preparative Example 1-1: Preparation of phenol-formaldehyde resin (resol polymer)

10 g of phenol was dissolved in a round-bottom flask at 40° C., and an aqueous solution of 20% by weight of NaOH (2.14 g) was added thereto. The resulting mixture was stirred for 10 minutes, and 17.2 g of a formaldehyde solution was added slowly, and the solution was then heated to 70° C. The mixture was reacted for an hour and adjusted to the neutral pH (pH 7.0) using 1 M HCl. Water was removed in a vacuum oven at 45° C. A proper amount of THF was added to form white NaCl, which was then filtered out. Then, the resol/THF was further dried at 45° C. under vacuum to prepare a resol polymer.

### Preparative Example 1-2: Preparation of poly(ethylene oxide)-block-poly(styrene) (PEO-b-PS)

PEO-b-PS was prepared by means of atomic transfer radical polymerization (ATRP).

To prepare an mPEO-Br macroinitiator, first of all, 10 g of mPEO-OH was dissolved in 50 mL of CH₂Cl₂ and an excessive amount of trimethylamine (3 mL). Under the ice-bath conditions, 0.74 mL of 2-bromoisobutylbromide was slowly added to a solution. The solution was stirred at room temperature for 8 hours. The yellowish solution was purified through extraction with H₂O and three-time precipitation with cold ether. The product was dried at room temperature under vacuum.

Next, to polymerize styrene, 1.27 g of mPEO-Br, 10 g of styrene, 0.036 g of CuBr, and 0.043 g of PMDETA were mixed in a 50-mL round-bottom flask. After three freeze-pump-thaw cycles, the solution was heated to 110° C., and stirred for 12 hours. The product was diluted with THF and filtered through a neutral alumina column (for removing a copper catalyst). To precipitate PEO-b-PS, the solution was filtered, and cold methanol was added thereto. Finally, the PEO-b-PS was dried at room temperature under vacuum.

### Preparative Example 1-3: Synthesis of Nanostructures

The resol polymer and PEO-b-PS prepared in Preparative Examples 1-1 and 1-2 were prepared. First, PEO-b-PS (number average molecular weight (Mₙ)=37 kg mol⁻¹, PEO 13.5% by weight) was dissolved in THF. Thereafter, 20% by weight of the resol polymer solution dissolved in THF was added to the PEO-b-PS solution. 68% by weight of nitric acid (in H₂O) and tetraethylorthosilicate (TEOS) were sequentially added. The PEO-b-PS, the resol, the THF, the nitric acid (in HNO₃), H₂O, and the TEOS were mixed at a mass ratio of 1:1.33:29:12.87:6:0.87, that is, at a weight ratio of 0.15 g:0.2 g:4.36 g:1.93 g:0.9 g:0.13 g. The solution was stirred for an hour and transferred to a Petri dish. The Petri dish was put on a hot plate at 50° C. in a fume hood and was uncovered while evaporating a solvent. A flat glass cover was used in a control experiment to regulate a solvent evaporation rate. After the solvent was evaporated, a BCP/inorganic hybrid film was annealed at 100° C. 12 to 16 hours in an oven. The assembled film was heated to 700° C. under an argon (a carbon/silica composite) condition, and to 500° C. in the air. In this case, the heat treatment was conducted at a heating rate of 1° C./min.

### Preparative Example 1-4: Conjugation of DNA to Pores of Nanostructures

A triplex DNA having a base sequence set forth in SEQ ID NO: 1 (5′-NH₂-TTA AAA GGG GGG TTT ACC CCC CTT TTC TTT GTT TTC CCC CCT TGG CC) was conjugated to nanostructures. This was done in two steps: i.e., modifying nanostructures with an amine and conjugating DNA to the nanostructures. To prepare nanostructures modified with an amine, the prepared nanostructures (50 mg) were dispersed in toluene (5 mL), 20 μL of (3-aminopropyl)triethoxysilane (APTES) was added thereto. Thereafter, the solution was stirred for 24 hours. The nanostructures were centrifuged at 13,200 rpm for 10 minutes and washed three times with methanol and water. An amount of the amine in the nanostructures was quantified using a fluorescamine assay. As a result, it was confirmed that the nanostructures contained an amine functional group at a concentration of 36.3 μmol/g. The nanostructures modified with the amine in this way were dispersed in HPLC-graded acetonitrile, and 5 equivalents of disuccinimidyl suberate (DSS) were added thereto. Then, the resulting solution was stirred for 30 minutes.

This suspension was centrifuged at 13,200 rpm for 10 minutes to remove unreacted DSS, and then re-dispersed in HPLC-graded acetonitrile. Thereafter, triplex DNA modified with 2 equivalents of amine was added thereto and reacted for 24 hours. The nanostructures to which the DNA was conjugated through the reactions were purified through centrifugation at 13,200 rpm for 10 minutes, and an amount of the DNA conjugated to the nanostructures was quantified using the optical density of DNA. It was confirmed that 2.91 μmol of DNA was conjugated to 1 g of the nanostructures.

### Preparative Example 2: Preparation of Nanoparticles

### Preparative Example 2-1: Synthesis of Gold Nanoparticles Encapsulated with Citric Acid

Gold nanoparticles were synthesized by reducing HAuCl₄ in the presence of citrate ions at a high temperature. A HAuCl₄ solution (20 mL, 1.5 mM in ultrapure water) was prepared while mild stirring at 120° C. After 15 minutes, 400 μL of a citrate solution (0.35 M in ultrapure water) was added to the HAuCl₄ solution. The color of the solution was changed from lemon yellow to dark red, and the solution was cooled at room temperature, and then stored at 4° C. without any further purification.

### Preparative Example 2-2: Conjugation of DNA to Surfaces of Gold Nanoparticles

I-motif DNA having a base sequence set forth in SEQ ID NO: 3 (5′-SH-TTT GGG TTA GGG TTA GGG TTA GCG), and DNA having a base sequence set forth in SEQ ID NO: 4 (5′-CCC TAA CCC TAA CCC TAA CCC), which was complementary to the i-motif DNA, were annealed together for an hour. After the annealing, the annealed i-motif DNA (50 nmol in 100 μL acetate buffer, pH=5.5) was modified with thiols at room temperature for an hour using 5 μL of 5 mM tris(2-carboxyethyl)phosphine hydrochloride (TCEP), and the triplex DNA having a base sequence set forth in SEQ ID NO: 2 (5′-SH-TTT TGG GCA AGG GGG GAA AT) was activated through modification with thiols in the same manner. The activated double-helical i-motif DNA was added to the previously prepared gold nanoparticles (13 nm) at a molar ratio of 1:100, and the activated triplex DNA was added at a molar ratio of 1:50. Subsequently, the initial mixture was incubated for 4 days in a saline aging process with a gradient of 0.1 M NaCl to 0.3 M NaCl. The final product was purified through three-time membrane centrifugation (MWCO: 30 k) using a Tris-acetate buffer of 0.1 M NaCl (pH=8.2) and stored in a Tris-acetate buffer of 0.3 M NaCl (pH=8.2).

### Preparative Example 3: Preparation of Nanostructures Supported with Nanoparticles

As the final product prepared in Preparative Example 2-2, the nanoparticles (5 nM) were dissolved in a Tris-acetate buffer including 0.3 M NaCl, and then added to a solution of the nanostructures (0.1 mg/mL) prepared in Preparative Example 1-4. The resulting mixture was slowly mixed for 10 minutes, sonicated for 10 minutes, and incubated at 2° C. for 30 minutes. The solution was centrifuged (at 4,000 rpm for 5 minutes) to form a pellet of nanostructures supported with nanoparticles (nanoparticle-loaded nanostructures), and unbound nanoparticles were separated off. An amount of the nanoparticles in the supernatant was quantified to determine an amount of the supported nanoparticles bound to the nanostructures using a back titration method.

### Preparative Example 4: Preparation of Nanostructures Supported with Nanoparticles and Doxorubicin

The nanostructures prepared in Preparative Example 3 were loaded with a drug. In the purified nanostructures, doxorubicin was added until its content reached 500 molar equivalents per one nanostructure, and then incubated for 12 to 16 hours. To purify an experimental solution, the nanostructures loaded with doxorubicin were centrifuged at 4,000 rpm for 5 minutes, and the fluorescence of doxorubicin in the supernatant was measured to quantify an amount of doxorubicin loaded onto the nanostructures using a back titration method. As a result, it was confirmed that one nanostructure was loaded with approximately 486 doxorubicin molecules.

### Example 1

The nanostructures supported with the nanoparticles prepared in Preparative Example 3.

### Example 2

The nanostructures supported with the nanoparticles and doxorubicin prepared in Preparative Example 4.

### Comparative Example 1

The nanostructures supported with the nanoparticles prepared in Preparative Example 2 by conjugating the nanostructures, which were prepared in Preparative Example 1 by conjugating the triplex DNA having a base sequence set forth in SEQ ID NO: 5 (5′-NH₂-TTA AAA AAA GAA AAG AAT TTA TTC TTT TCT TCT TTG TTT TCT TCC GG) instead of SEQ ID NO: 1, to DNAs of SEQ ID NO: 6 (5′-SH-TTT TCC GGA AGA AAA CAA AG), SEQ ID NO: 7 (5′-SH-TTT TAG TCA AAT TGC AAT ACC TCG), and SEQ ID NO: 8 (5′-CGA GGT AAT GCA ATT TGA CTA) instead of SEQ ID NO: 2, SEQ ID NO: 3, and SEQ ID NO: 4, respectively.

### Comparative Example 2

The nanostructures supported with the nanoparticles prepared in Preparative Example 2 by conjugating the nanostructures prepared in Preparative Example 1 to DNAs of SEQ ID NO: 6, SEQ ID NO: 7, and SEQ ID NO: 8 instead of SEQ ID NO: 2, SEQ ID NO: 3, and SEQ ID NO: 4, respectively.

### Experimental Example 1: TEM and SEM Images of Nanostructures

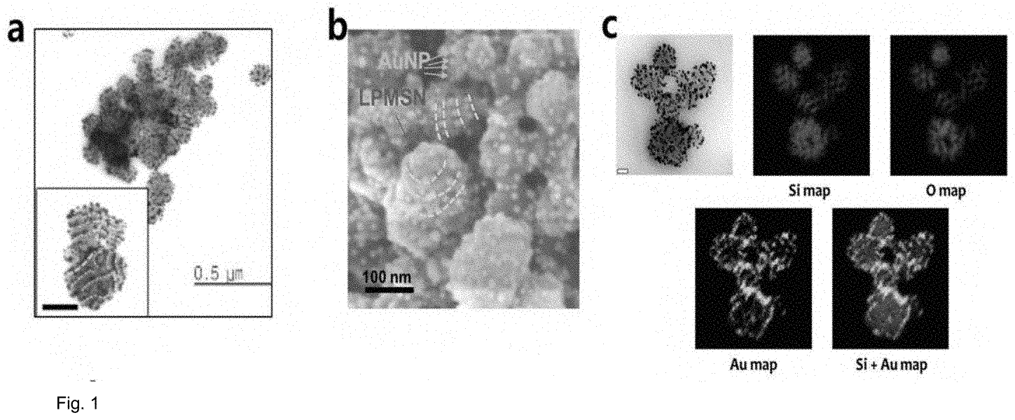

It was confirmed that the nanoparticles were supported in the nanostructures prepared in Example 1 using TEM and SEM (FIG. 1).

More specifically, FIG. 1A is a TEM image of the nanostructures of Example 1, confirming that the nanoparticles are supported in the nanostructures, and FIG. 1B is an SEM image of the nanostructures of Example 1, confirming that the nanoparticles are supported through the interior of the nanostructures.

As shown in FIGS. 1A and 1B, it was also confirmed that the nanostructures had a diameter of approximately 150 nm, and the nanoparticles had a diameter of approximately 13 nm.

Also, FIG. 1C shows the electron energy loss spectroscopy (EELS) analysis results of the nanostructures of Example 1, confirming that the nanoparticles composed of gold (Au) are supported in the nanostructures composed of silica components (Si and O). As shown in FIG. 1C, it was confirmed that the nanostructures were actually distinguished from the nanoparticles supported therein.

Accordingly, it was confirmed that the nanostructures according to the present invention had the nanoparticles supported therein.

### Experimental Example 2: Evaluation of Release of Nanoparticles and Drug According to pH Change

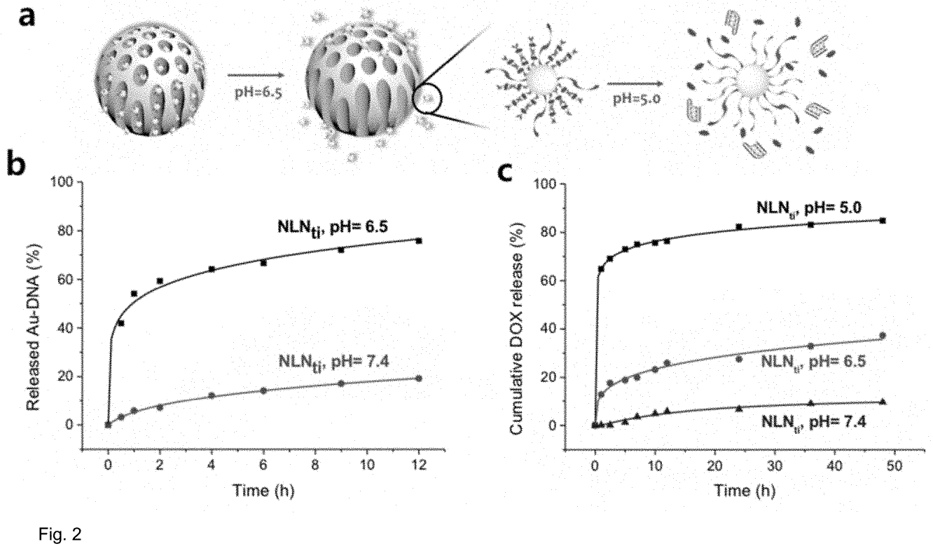

To check whether the nanostructures of Example 2 function to sequentially release the nanoparticles and the drug according to the pH gradient of the cancer tissues, their release behaviors were determined through absorbance and fluorescence. The results are shown in FIG. 2.

Specifically, the nanostructures of Example 2 were well dispersed in a Tris-acetate buffer containing 0.3 M NaCl. To check the release behavior of nanoparticles, 1 M HCl was added to the nanostructures solution of Example 2 so that the pH of the nanostructures solution was adjusted to pH 6.5, violently mixed, and then centrifuged (at 4,000 rpm for 5 minutes). An amount of the nanoparticles released from the nanostructures of Example 2 was calculated based on the optical density of the supernatant at 520 nm. The results are shown in FIG. 2B. Meanwhile, to evaluate the release of doxorubicin from the nanoparticles, 1 M HCl was added to the nanostructures solution of Example 2 so that the pH of the nanostructures solution was sequentially adjusted to pH 6.5 and pH 5.0. Then, the solution whose pH was adjusted to pH 6.5 and the solution whose pH was adjusted to pH 5.0 were violently mixed and centrifuged (at 13,200 rpm for 15 minutes). Subsequently, each of the supernatants was taken to measure an amount of doxorubicin released from the nanostructures of Example 2. In this case, the amount of the released doxorubicin was calculated according to the following equation by measuring the fluorescence of doxorubicin (excitation at 495 nm, and emission at 555 nm). This calibration curve is shown in FIG. 2C.

Amount of Doxorubicin Released=(Amount of Doxorubicin Released)/(Loading Dose of doxorubicin)×100 Equation:

FIG. 2A is a schematic diagram showing that the nanostructures of Example 2 function to sequentially release the nanoparticles and the drug depending on the pH gradient, and FIG. 2B shows the results of confirming the release behaviors of the nanoparticles and the drug from the nanostructures of Example 2 depending on the time and pH level.

As a result, it was confirmed that the nanoparticles were selectively released from the nanostructures of Example 2 at pH 6.5, and also confirmed that doxorubicin was selectively released from the nanostructures of Example 2 at pH 5.

Based on these results, it was confirmed that the nanostructures according to the present invention was able to intelligently control the nanoparticle or drug release behavior in response to different pH levels, depending on the control of a base sequence.

### Experimental Example 3: Evaluation of Penetration of Nanostructures Using 3D Spheroid Model

Spheroids of MDA-MB-231 cells were transferred to 96-well plates containing 90 μL of fresh media whose pH was adjusted to pH 7.4 and pH 6.5, respectively. Each of the nanostructures of Example 1 and the nanostructure solution of Comparative Example 1, both of which were tagged with Cy3 and Cy5, was added at each pH level to the spheroids of MDA-MB-231 cells and incubated for 6 hours. As the control, each of the nanoparticles prepared in Preparative Example 2 and the nanostructures solution prepared in Preparative Example 1, both of which were tagged with Cy3 and Cy5, was equally added to the spheroids of MDA-MB-231 cells at each pH level and incubated for 6 hours.

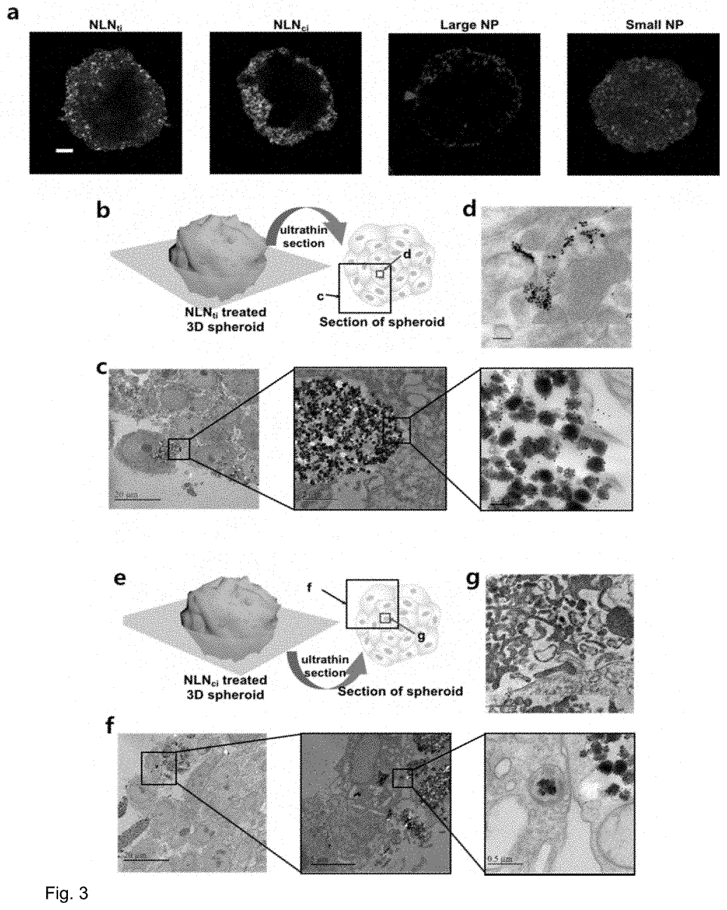

After the incubation was completed, the spheroids of MDA-MB-231 cells from all the experimental groups were washed three times with DPBS, and single cells were treated with trypsin, and then re-dispersed in DPBS. The cells were analyzed according to the protocol provided by the manufacturer using FACS Calibur (Becton Dickinson) and BD Cell Quest software (Becton Dickinson). The results are shown in FIG. 3A.

Also, FIG. 3B is a schematic diagram showing the nanostructures of Example 1, and FIG. 3E is a schematic diagram showing the spheroids of MDA-MB-231 cells treated with the nanostructures of Comparative Example 1. Here, the arrows indicate regions shown in FIGS. 3C, 3D, 3F, and 3G.

FIG. 3C is a TEM image of the spheroids of MDA-MB-231 cells treated with the nanostructures of Example 1, and FIG. 3D is a TEM image of an enlarged internal region of the spheroids of MDA-MB-231 cells treated with the nanostructures of Example 1.

Meanwhile, FIG. 3F is a TEM image of the spheroids of MDA-MB-231 cells treated with the nanostructures of Comparative Example 1, and FIG. 3G is a TEM image of an enlarged internal region of the spheroids of MDA-MB-231 cells treated with the nanostructures of Comparative Example 1.

As can be seen from FIG. 3, it was confirmed that the nanostructures of Example 1 and the nanoparticles prepared in Preparative Example 2 easily penetrated into the 3D spheroids. However, it was confirmed that the nanostructures of Comparative Example 1 and the nanostructures prepared in Preparative Example 1 remained on the surfaces of the spheroids without penetrating deep into the spheroids.

Here, it was confirmed that, because the pH-sensitive DNA was conjugated to the nanostructures of Example 1 so that the nanoparticles were released from the nanostructures, the nanoparticles had an effect of penetrating deep into the spheroids. Therefore, it was confirmed that the nanostructures according to the present invention responded to the pH levels according to the control of a base sequence, thereby inducing the cancer tissue-specific inflow to maximize the delivery efficiency.

### Experimental Example 4: Confirmation of Deep Penetration of Nanostructures in Bio-Distribution and Cancer Tissues upon Injection through Tail Vein in Animal Model

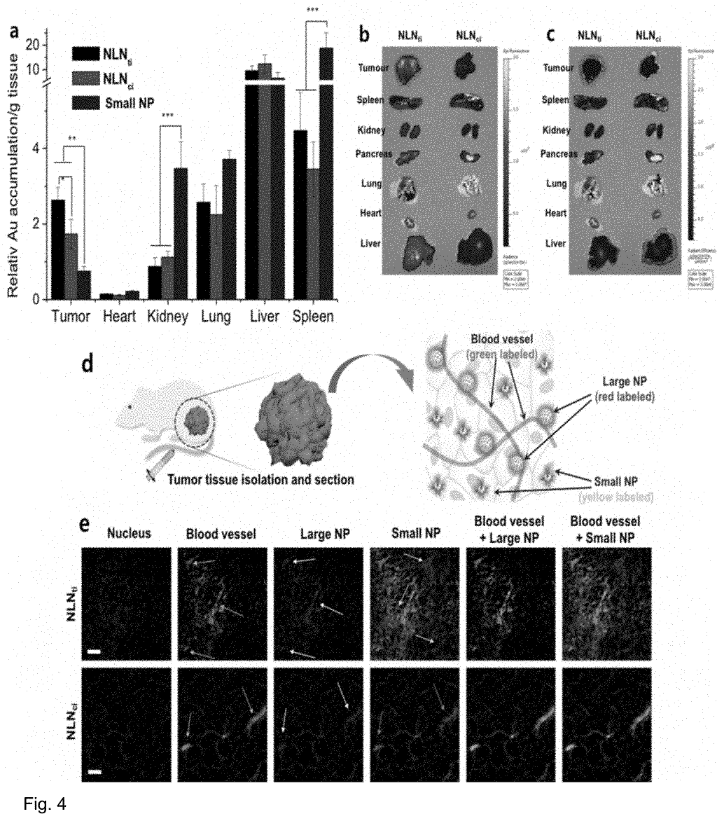

To prepare an animal model, first of all, MDA-MB-231 cells were subcutaneously administered to the flanks of 6-week-old female Balb/c-nu/nu mice at a concentration of 3×10⁷ cells/mouse. After the administration, when the average tumor volume reached 300 mm³, the mice were randomly divided to three groups (n=3 per group), and 200 μL of each of the nanoparticles prepared in Example 1, Comparative Example 1 and Preparative Example 2, which were tagged with Cy3 and Cy5, administered through tail veins of the mice. After 24 hours, the mice were sacrificed, and fluorescence images of respective organs for Cy3 and Cy5 channels were obtained and analyzed using an IVIS Spectrum in vivo imaging system for small animals. The results are shown in FIG. 4. Here, Cy3 was used to check the nanoparticles, and Cy5 was used to check the nanostructures.

As shown in FIGS. 4A to 4C, it was confirmed that a larger amount of the nanostructures of Example 1 and Comparative Example 1 were selectively distributed in the cancer tissues, compared to the nanoparticles prepared in Preparative Example 2, and also confirmed that a higher concentration of the nanoparticles accumulated in the cancer tissues in the case of the nanostructures of Example 1, compared to the nanostructures of Comparative Example 1.

FIG. 4E is an image showing the distributions of the nanostructures of Example 1 and Comparative Example 1 in the cancer tissues after blood vessels are tagged with an antibody in tissue slices obtained by microtoming a cancer tissue. Here, blood vessels were stained with tomato lectin-FITC (green), and the nuclei were stained with 2-(4-amidinophenyl)-1H-indole-6-carboxamidine (DAPI, blue). The nanostructures were tagged with Cy5 (red), and the nanoparticles were tagged with i-motif DNA-labeled Cy3 (yellow). A green arrow indicates a blood vessel in a tissue. A white arrow indicates nanostructures around the blood vessel. A yellow arrow indicates a high reperfusion of the nanoparticles released from the nanostructures of Example 1. A blue arrow indicates a low perfusion of the nanoparticles released from the nanostructures of Comparative Example 1. Scale bar represents 50 μm.

As can be seen from FIG. 4E, it was confirmed that the nanostructures of Example 1 capable of releasing the nanoparticles enabled the nanoparticles to penetrated deep into the sites distributed far from the blood vessels.

Accordingly, it was confirmed that the nanostructures according to the present invention also induced the cancer tissue-specific inflow of the nanoparticles, thereby maximizing the delivery efficiency and minimizing the side effects.

### Experimental Example 5: Evaluation of Anti-Cancer Effect of Nanostructures

### 1) Two-Dimensional (2D) Evaluation of Anti-Cancer Effect

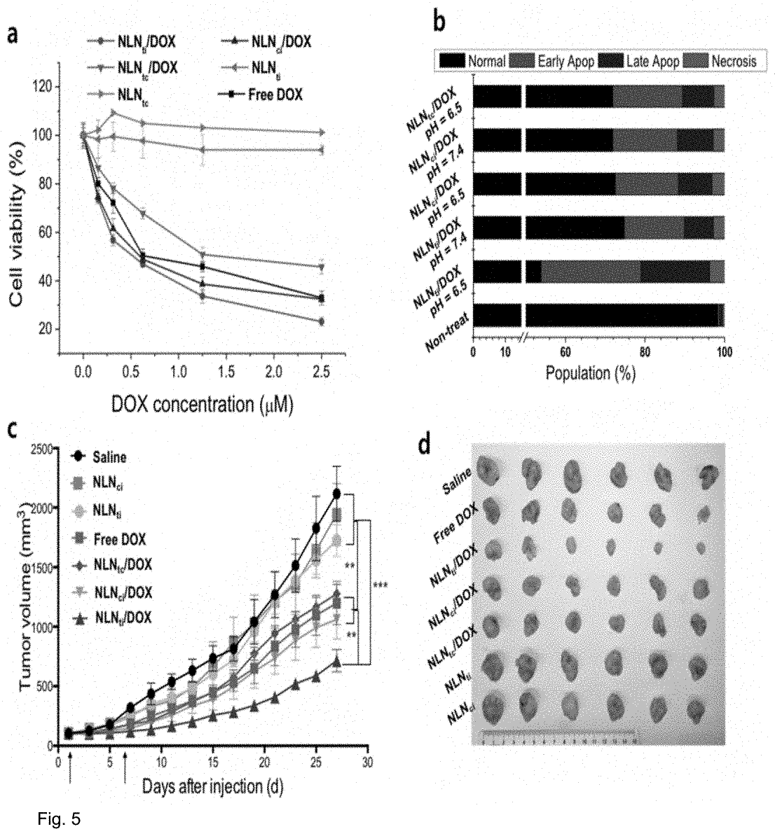

To evaluate the 2D cell viability, MDA-MB-231 cells were seeded in a 96-well plate at a density of 6,000 cells/well, and then incubated overnight. Each of varying concentrations of doxorubicin, and the solutions of Example 2, Comparative Example 1 in which doxorubicin was supported, Comparative Example 2 in which doxorubicin was supported, Example 1, and Comparative Example 1 was treated with a fresh medium and incubated for another 48 hours. After the incubation, the MDA-MB-231 cells were washed with DPBS, and the media was replaced with 200 μL of a MTT-containing solution (0.5 mg/mL). After the MDA-MB-231 cells were incubated for 4 hours in a CO₂ incubator, the media were removed, and 200 μL of DMSO was added thereto. 100 μL of each of the solutions was transferred to a new 96-well plate and measured for optical density at 570 nm using a microplate reader. The results are shown in FIG. 5A.

### 2) 3D Evaluation of Anti-Cancer Effect

For evaluation of 3D cell viability or toxicity, spheroids were transferred to a 96-well plate containing 90 μL of a fresh medium (pH: 7.4 or 6.5). The spheroids were treated with each of the solutions of Example 2 in which a final concentration of doxorubicin was adjusted to 1 μM at different pH levels, Comparative Example 1 in which doxorubicin was supported, and Comparative Example 2 in which doxorubicin was supported, and then incubated for another 24 hours. After the incubation, the spheroids were washed with DPBS, and single cells were treated with trypsin. Subsequently, the cell viability was tested using an Annexin V-FITC apoptosis detection kit. Here, a total of 10,000 cells in each experimental group were counted according to the protocol provided in the flow cytometry. The spheroids not treated with trypsin were used to determine a critical value for Annexin V-FITC and propidium iodine staining methods. The results are shown in FIG. 5B.

### 3) Evaluation of Anti-Cancer Effect Using Animal Model

To prepare an animal model, first of all, MDA-MB-231 cells were subcutaneously administered to the flanks of 6-week-old female Balb/c-nu/nu mice at a concentration of 3×10⁷ cells/mouse. After the administration, when the average tumor volume reached 300 mm³, the mice were randomly divided to seven groups (n=7 per group), and 200 μL of each of physiological saline, doxorubicin, and the nanoparticles of Example 1, Example 2, Comparative Example 1, Comparative Example 1 in which doxorubicin was supported, and Comparative Example 2 was administered to the tail veins of the mice one a day at a doxorubicin concentration of 5 mg/kg for 6 days. The anti-cancer effect was evaluated by monitoring the volumes of tumors. Each of the tumors was measured in a 2D manner every two days using electronic calipers. The tumor volumes were calculated according to the equation for prolellate spheroids.

Tumor Volume=ab²/2

wherein “a” represents the longest length of a spheroid, and “b” represents the shortest length of the spheroid. After the initial administration, the volume of tumor was monitored for 27 days. After the monitoring was completed, all the mice were sacrificed to measure the weights of the tumors. All the results are indicated by an average±standard deviation, and a statistical difference was proved by one- or two-way ANOVA using GraphPad Prism software 6. The results are shown in FIGS. 5C and 5D.

As can be seen from FIG. 5, it was confirmed that, because the 2D evaluation results of the anti-cancer effect did not require the deep penetration, there was hardly a difference in apoptotic effects in cancer cells in the case of Example 2 in which doxorubicin was supported, Comparative Example 1, and Comparative Example 2. However, the 3D evaluation results of the anti-cancer effect showed that the early apoptosis, late apoptosis, and necrosis occurred more actively at pH 6.5, compared to those at pH 7.4 in the case of Example 2. Meanwhile, Comparative Examples 1 to 2 showed substantially similar apoptotic and necrotic effects at pH 7.4 and pH 6.5. Likewise, the evaluation of the animal model showed that the nanostructures of Example 2 had a remarkably superior anti-cancer effect because the nanostructures of Example 2 penetrated deeper into cancer tissues, compared to those of Comparative Example 1.

Accordingly, it was confirmed that the nanostructures according to the present invention was able to penetrate deep into tissues depending on the pH environments in a substantial 3D model, and thus can be used as the drug delivery systems or nano-drugs because the nanostructures have high drug delivery efficiency and selectivity.

1. Nanostructures having nanoparticles supported therein,
wherein the nanoparticles and the nanostructures have pH sensitivity, and
each of the nanoparticles and the nanostructures comprise DNA.

2. The nanostructures of claim 1, wherein the nanoparticles have a diameter of 20 nm or less, and the nanostructures have a diameter of 50 nm or more.

3. The nanostructures of claim 1, wherein the DNA comprises a base sequence in which a ratio of cytosine (C) is in a range of 40% to 80%.

4. The nanostructures of claim 1, wherein the nanoparticles and the nanostructures are complementarily bound by means of the base sequence of the DNA.

5. The nanostructures of claim 1, wherein the nanostructures comprise DNA having a triplex base sequence, and
the nanoparticles comprise DNA having a sequence complementary to the triplex base sequence.

6. The nanostructures of claim 5, wherein the triplex base sequence is a base sequence set forth in SEQ ID NO: 1, and
the sequence complementary to the triplex base sequence is a base sequence set forth in SEQ ID NO: 2.

7. The nanostructures of claim 1, wherein the nanostructures have pH sensitivity at pH 6.0 to 6.5.

8. The nanostructures of claim 1, wherein the nanoparticles have pH sensitivity at pH 5.5 or lower.

9. The nanostructures of claim 1, wherein an i-motif DNA base sequence and a sequence complementary thereto are conjugated onto surfaces of the nanoparticles.

10. The nanostructures of claim 9, wherein the i-motif DNA base sequence is a base sequence set forth in SEQ ID NO: 3, and
the sequence complementary to the i-motif DNA base sequence is a base sequence set forth in SEQ ID NO: 4.

11. The nanostructures of claim 9, wherein an anticancer drug is supported in the i-motif DNA base sequence and the sequence complementary thereto.

12. The nanostructures of claim 11, wherein the anticancer drug comprises one or more selected from the group consisting of doxorubicin (DOX), cisplatin, etoposide, paclitaxel, doxetaxel, fluoropyrimidine, oxaliplatin, campthotecan, belotecan, podophyllotoxin, vinblastine sulfate, cyclophosphamide, actinomycin, vincristine sulfate, methotrexate, bevacizumab, thalidomide, erlotinib, gefitinib, camptothecin, tamoxifen, anasterozole, Gleevec, 5-fluorouracil (5-FU), floxuridine, leuprolide, flutamide, zoledronate, vincristine, gemcitabine, streptozotocin, carboplatin, topotecan, irinotecan, vinorelbine, hydroxyurea, valrubicin, retinoic acid, meclorethamine, chlorambucil, busulfan, doxifluridine, vinblastin, mitomycin, prednisone, testosterone, mitoxantron, aspirin, salicylates, ibuprofen, naproxen, fenoprofen, indomethacin, phenylbutazone, cyclophosphamide, mechlorethamine, dexamethasone, prednisolone, celecoxib, valdecoxib, nimesulide, cortisone, corticosteroid, and a combination thereof.
